(19) United States
(12) Patent Application Publication
(10) Pub. No.: US 2023/0017988 A1
(43) Pub. Date: Jan. 19, 2023

(54) TREATMENT INSTRUMENT, TREATMENT SYSTEM AND TREATMENT METHOD

(71) Applicant: OLYMPUS CORPORATION, Tokyo (JP)

(72) Inventors: Satoshi HONDA, Tokyo (JP); Gen KATO, Tokyo (JP)

(73) Assignee: OLYMPUS CORPORATION, Tokyo (JP)

(21) Appl. No.: 17/946,267

(22) Filed: Sep. 16, 2022

Related U.S. Application Data
(63) Continuation of application No. PCT/JP2020/033136, filed on Sep. 1, 2020.

(51) Int. Cl. A61B 18/14

(52) U.S. Cl. CPC A61B 18/1445; A61B 2018/00994

(57) ABSTRACT

A treatment instrument includes: a sheath; and an end effector that is provided at a distal end of the sheath. The end effector is capable of gripping a living tissue and applying high frequency energy to the living tissue. The end effector includes a treatment surface, and a pair of electrodes that can grip the living tissue and apply the high frequency energy to the living tissue. The treatment surface includes a distal end area that is provided on a distal end side of the treatment surface, and another area that is provided on a proximal end side of the treatment surface. The distal end area applies, to the living tissue, high frequency energy that is higher than high frequency energy applied by the other area. One of the pair of electrodes includes a distal end portion and a proximal end portion.

### CROSS-REFERENCE TO RELATED APPLICATION

This application is a continuation of International Application No. PCT/JP2020/033136, filed on Sep. 1, 2020, the entire contents of which are incorporated herein by reference.

### BACKGROUND

### 1. Technical Field

The present disclosure relates to a treatment instrument, a treatment system, and a treatment method.

### 2. Related Art

In the related art, there is a known treatment instrument that performs treatment on a region targeted for treatment of a living tissue (hereinafter, referred to as a target region) by applying high frequency energy to the target region.

A known treatment instrument includes an end effector that grips the target region and that applies high frequency energy to the target region in accordance with supplied electrical power. The end effector includes a pair of jaw members that are relatively rotatable about a pivot pin provided on a proximal end side. Then, the pair of jaw members grip the target region by being opened and closed in accordance with the relative rotation about the pivot pin.

### SUMMARY

In some embodiments, a treatment instrument includes: a sheath extending along a central axis from a distal end to a proximal end; and an end effector that is provided at the distal end of the sheath. The end effector can grip a living tissue and apply high frequency energy to the living tissue in accordance with supplied electrical power to perform treatment on the living tissue. The end effector includes a treatment surface for performing treatment on the living tissue, and a pair of electrodes that can grip the living tissue and apply the high frequency energy to the living tissue in accordance with the supplied electrical power. The treatment surface includes a distal end area that is provided on a distal end side of the treatment surface and that includes a distal end of the end effector, and another area that is provided on a proximal end side of the treatment surface. The distal end area is configured to apply, to the living tissue, high frequency energy that is higher than high frequency energy applied by the other area, and a first one of the pair of electrodes includes a distal end portion that is located in the distal end area and a proximal end portion that is located in the other area.

In some embodiments, a treatment system includes: a treatment instrument for performing treatment on a living tissue; and a control device for controlling an operation of the treatment instrument. The treatment instrument includes a sheath, and an end effector that is provided at a distal end of the sheath. The end effector can grip the living tissue and apply high frequency energy to the living tissue in accordance with electrical power that is supplied from the control device to perform treatment on the living tissue, and the end effector includes a treatment surface for performing treatment on the living tissue, and a pair of electrodes that can apply the high frequency energy to the living tissue. The treatment surface includes a distal end area that is provided on a distal end side of the treatment surface and that includes a distal end of the end effector, and another area that is provided on a proximal end side of the treatment surface. The distal end area is configured to apply, to the living tissue, high frequency energy that is higher than high frequency energy applied by the other area, and a first one of the pair of electrodes includes a distal end portion that is located in the distal end area and a proximal end portion that is located in the other area.

In some embodiments, provided is a method of performing treatment on a living tissue by controlling electrical power supplied to an end effector that can grip the living tissue, and includes a pair of electrodes that can apply high frequency energy to the living tissue. A first one of the pair of electrodes includes a distal end portion located in a distal end area and a proximal end portion located in another area. The method includes: applying, to the living tissue gripped by the distal end portion, energy that is higher than energy supplied to the living tissue gripped by the proximal end portion, to perform treatment on the living tissue.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an exemplary embodiment;

FIG. 2 is a cross-sectional view illustrating a transducer unit;

FIG. 3 is a diagram illustrating a configuration of a jaw and a vibration transmission member;

FIG. 4 is a diagram illustrating a configuration of the jaw and the vibration transmission member;

FIG. 5 is a diagram illustrating a configuration of the jaw and the vibration transmission member;

FIG. 6 is a perspective view illustrating the vibration transmission member;

FIG. 7 is a diagram illustrating a configuration of an end effector according to an exemplary embodiment;

FIG. 8 is a diagram illustrating a configuration of an end effector according to an exemplary embodiment;

FIG. 9 is a diagram illustrating a configuration of an end effector according to an exemplary embodiment; and

FIG. 10 is a diagram illustrating a configuration of an end effector according to an exemplary embodiment.

### DETAILED DESCRIPTION

Modes (hereinafter, embodiments) for carrying out the disclosure will be described below with reference to the drawings. Furthermore, the disclosure is not limited to the embodiments described below. In addition, in description of the drawings, components that are identical to those in drawings are assigned the same reference numerals.

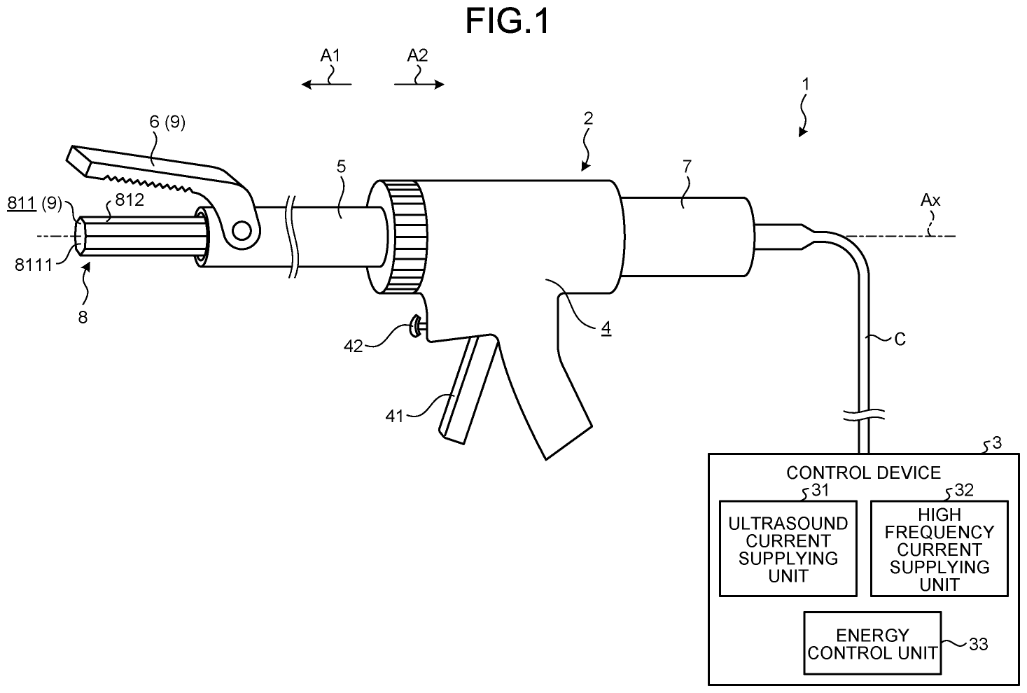

Schematic configuration of treatment system FIG. 1 is a diagram illustrating a treatment system 1 according to an exemplary embodiment.

The treatment system 1 performs treatment on a region targeted for treatment of a living tissue (hereinafter, referred to as a target region) by applying ultrasound energy and high frequency energy to the target region. Here, the treatment indicates, for example, coagulation and incision of the target region. The treatment system 1 includes, as illustrated in FIG. 1, an ultrasound treatment instrument 2 and a control device 3.

The ultrasound treatment instrument 2 is a medical treatment instrument using, for example, a bolt-clamped Langevin-type transducer (BLT) for performing treatment on the target region through an abdominal wall. The ultrasound treatment instrument 2 includes, as illustrated in FIG. 1, a handle 4, a sheath 5, a jaw 6, a transducer unit 7, and a vibration transmission member 8.

The handle 4 is a portion held by a hand of an operator. In addition, as illustrated in FIG. 1, the handle 4 is provided with an operation knob 41 and an operation button 42.

The sheath 5 has a cylindrical shape. Furthermore, in a description below, the central axis of the sheath 5 is referred to as a central axis Ax (FIG. 1). In addition, in a description below, one of the sides along the central axis Ax is referred to as a distal end side A1 (FIG. 1), whereas the other of the sides is referred to as a proximal end side A2 (FIG. 1). Then, the sheath 5 is attached to the handle 4 by inserting a part of the proximal end side A2 from the distal end side A1 of the handle 4 into the interior of the handle 4.

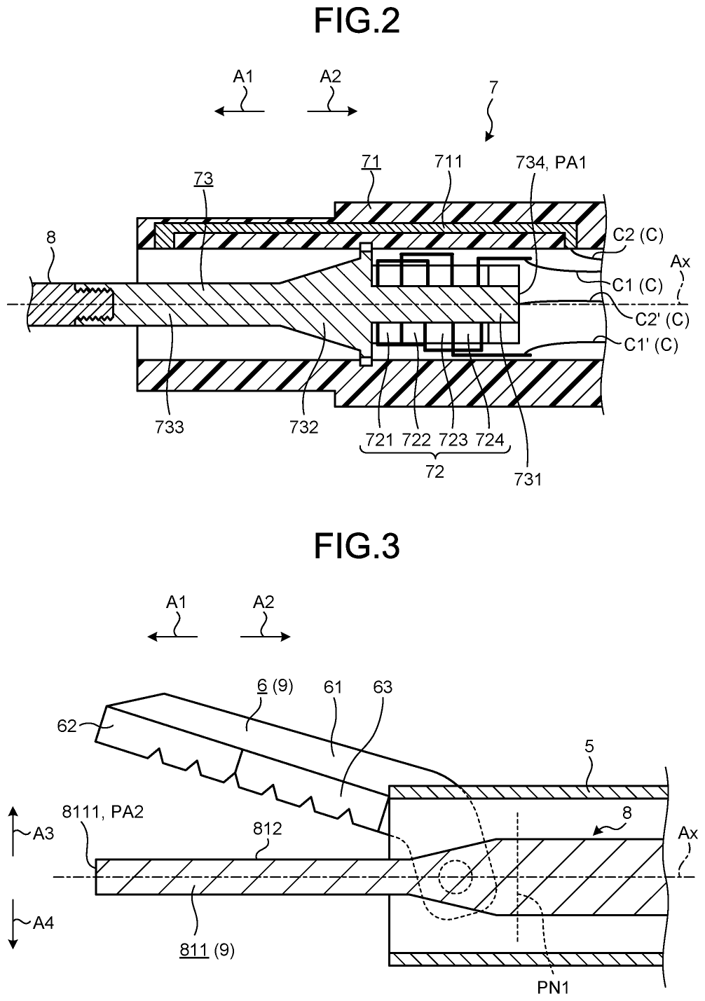

FIG. 2 is a cross-sectional view illustrating the transducer unit 7. Specifically, FIG. 2 is a cross-sectional view obtained by cutting the transducer unit 7 by a plane including the central axis Ax.

The transducer unit 7 includes, as illustrated in FIG. 2, a transducer case 71, an ultrasound transducer 72, and a horn 73.

The transducer case 71 linearly extends along the central axis Ax and is attached to the handle 4 by inserting a part of the transducer case 71 on the distal end side A1 from the proximal end side A2 of the handle 4 into the interior of the handle 4. Then, if the transducer case 71 is attached to the handle 4, the end portion thereof on the distal end side A1 is coupled to the end portion of the proximal end side A2 of the sheath 5.

The ultrasound transducer 72 is accommodated in the interior of the transducer case 71 and produces ultrasound vibration under the control of the control device 3. In the present embodiment, the ultrasound transducer 72 is a BLT that includes a plurality of piezoelectric elements 721 to 724 that are laminated along the central axis Ax. Furthermore, in the present embodiment, the piezoelectric elements are constituted by the four piezoelectric elements 721 to 724; however, the number of piezoelectric elements is not limited to four and the other number of piezoelectric elements may be provided.

The horn 73 is accommodated in the interior of the transducer case 71 and increases the amplitude of the ultrasound vibration produced by the ultrasound transducer 72. The horn 73 has a long shape that linearly extends along the central axis Ax. Furthermore, as illustrated in FIG. 2, the horn 73 is configured such that a first mounting portion 731, a cross-sectional area changing portion 732, and a second mounting portion 733 are arrayed from the proximal end side A2 toward the distal end side A1.

The first mounting portion 731 is a portion in which the ultrasound transducer 72 is mounted.

The cross-sectional area changing portion 732 is a portion that has a tapered shape such that the cross-sectional area of the cross-sectional area changing portion 732 is gradually decreased toward the distal end side A1 and is a portion that increases the amplitude of the ultrasound vibration.

The second mounting portion 733 is a portion in which the vibration transmission member 8 is mounted.

The jaw 6 and the vibration transmission member 8 is a portion that grips the target region and performs treatment on the target region by applying the ultrasound energy and the high frequency energy on the target region.

Furthermore, detailed configurations of the jaw 6 and the vibration transmission member 8 will be described later.

The control device 3 is electrically connected to the ultrasound treatment instrument 2 by an electric cable C (FIG. 1) and performs overall control of the operation of the ultrasound treatment instrument 2.

Furthermore, a detailed configuration of the control device 3 will be described later.

Configuration of Jaw and Vibration Transmission Member

In the following, detailed configurations of the jaw 6 and the vibration transmission member 8 will be described.

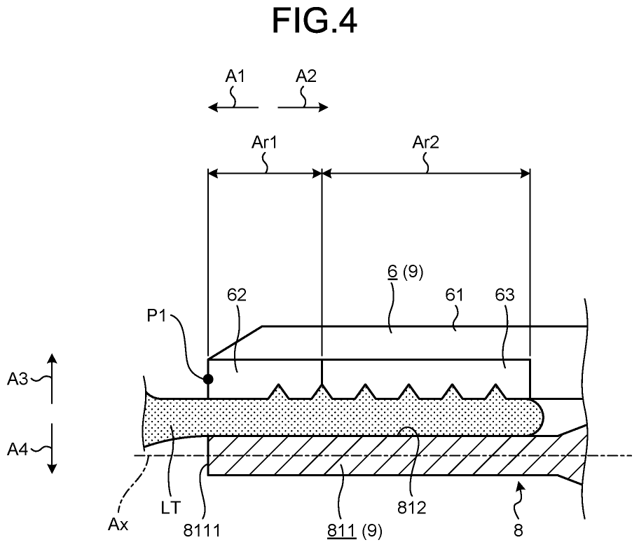

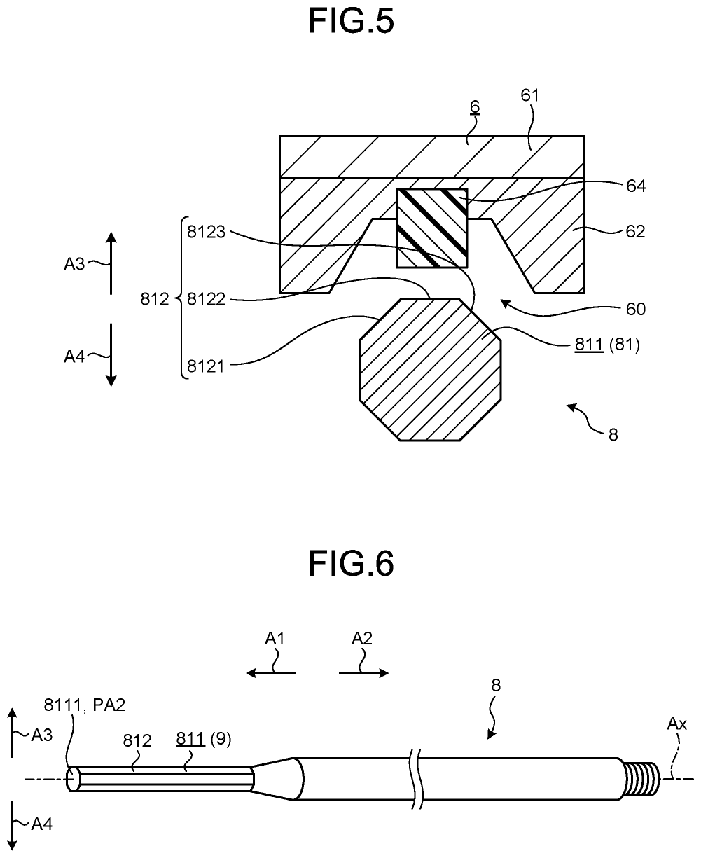

FIG. 3 to FIG. 5 are diagrams each illustrating the configurations of the jaw 6 and the vibration transmission member 8. FIG. 6 is a perspective view illustrating the vibration transmission member 8. Specifically, FIG. 3 is a cross-sectional view obtained by cutting the jaw 6 and the vibration transmission member 8 by a plane that includes the central axis Ax and that passes through a first treatment surface 812. FIG. 4 is a cross-sectional view obtained in the same way as that of FIG. 3 and indicates a state in which a target region LT is gripped by the jaw 6 and the vibration transmission member 8. FIG. 5 is a cross-sectional view obtained by cutting the jaw 6 and the vibration transmission member 8 by a plane that is perpendicular to the central axis Ax and that passes through a treatment portion 811.

Furthermore, in the following description of the configurations of the jaw 6 and the vibration transmission member 8, a side on which the jaw 6 is disposed (an upper side of FIG. 3 to FIG. 6) is referred to as an upper side A3 (FIG. 3 to FIG. 6), whereas a side on which the vibration transmission member 8 is disposed (a lower side of FIG. 3 to FIG. 6) is referred to as a lower side A4 (FIG. 3 to FIG. 6).

As illustrated in FIG. 6, the vibration transmission member 8 is formed of a conductive property material made of, for example, a titanium alloy, metallic glass, extra super duralumin (A7075), or the like and has a long shape that linearly extends along the central axis Ax. As illustrated in FIG. 1, the vibration transmission member 8 is inserted into the interior of the sheath 5 in a state in which the portion of the vibration transmission member 8 on the distal end side A1 externally extends. Furthermore, as illustrated in FIG. 2, the end portion of the vibration transmission member 8 on the proximal end side A2 is connected to the second mounting portion 733. Then, the vibration transmission member 8 performs treatment on the target region LT by transmitting the ultrasound vibration that is produced by the ultrasound transducer 72 that passes through the horn 73 from the end portion of the vibration transmission member 8 on the proximal end side A2 to the end portion of the vibration transmission member 8 on the distal end side A1 and applying the ultrasound vibration to the target region LT that is gripped between the end portion of the vibration transmission member 8 on the distal end side A1 and the jaw 6. In other words, treatment is performed on the target region LT by applying the ultrasound energy from the end portion of the distal end side A1.

Here, the vibration transmission member 8, the horn 73, and the ultrasound transducer 72 act as a single vibrating body that performs longitudinal vibration caused by the ultrasound vibration having a predetermined resonance frequency generated by the ultrasound transducer 72. Therefore, a proximal end surface 734 (FIG. 2) of the horn 73 is located at a most proximal end antinode position PA1 (FIG. 2) that is located at a position closest to the proximal end side A2 out of the antinode positions of the longitudinal vibration. Furthermore, a distal end surface 8111 (FIG. 3, FIG. 4, and FIG. 6) of the vibration transmission member 8 is located at a most distal end antinode position PA2 (FIG. 3, FIG. 4, and FIG. 6) that is located at a position closest to the distal end side A1 out of the antinode positions of the longitudinal vibration. Furthermore, the longitudinal vibration have a wave with the frequency of, for example, 47 kHz and the amplitude of, for example, 80 μm at the most distal end antinode position PA2.

In the vibration transmission member 8, the end portion thereof on the distal end side A1 functions as the treatment portion 811 (FIG. 1, and FIG. 3 to FIG. 6) that performs treatment on the target region LT in the state in which the target region LT is gripped between the jaw 6. The treatment portion 811 is a portion located closer to the distal end side A1 than the most distal end node position PN1 (FIG. 3) that is located at a position closest to the distal end side A1 out of the node position of the longitudinal vibration. Furthermore, although not specifically illustrated in the drawings, at the most distal end node position PN1, a lining for supporting the vibration transmission member 8 with respect to the sheath 5 is provided between the vibration transmission member 8 and the sheath 5. Furthermore, a part of the treatment portion 811 is in a state in which the part of the treatment portion 811 protrudes from the sheath 5 toward the distal end side A1.

In the present embodiment, as illustrated in FIG. 5, the treatment portion 811 has an octagon shape in cross section and is disposed in an orientation such that three sides 8121 to 8123 of the octagon shape in cross section face the upper side A3. Furthermore, the surface corresponding to the three sides 8121 to 8123 is the first treatment surface 812 that comes into contact with the target region LT in a state in which the target region LT is gripped between the treatment portion 811 and the jaw 6 and that performs treatment on the target region LT.

The jaw 6 is attached to the end portion of the sheath 5 on the distal end side A1 in a rotatable manner and grips the target region LT with the treatment portion 811. Then, the jaw 6 and the treatment portion 811 correspond to an end effector 9. In addition, in the interior of the handle 4 and the sheath 5, an opening/closing mechanism (not illustrated) that opens and closes the jaw 6 with respect to the treatment portion 811 in accordance with an operation of the operation knob 41 performed by the operator is provided. The jaw 6 includes, as illustrated in FIG. 3 to FIG. 5, a base 61, a distal end portion 62, and a proximal end portion 63 (FIG. 3 and FIG. 4).

The base 61 is constituted by a conductive property material and has a long shape that extends along the central axis Ax. Furthermore, the base 61 is attached such that the end portion of the base 61 on the proximal end side A2 is attached to the end portion of the sheath 5 on the distal end side A1 in a rotatable manner while supporting the distal end portion 62 and the proximal end portion 63.

Each of the distal end portion 62 and the proximal end portion 63 is provided on the surface of the base 61 at a position that faces the treatment portion 811. Specifically, the distal end portion 62 and the proximal end portion 63 are disposed at the positions indicated below.

Here, as illustrated in FIG. 4, in the state in which the target region LT is gripped by the end effector 9, an area of the distal end side A1 including a distal end P1 of the end effector 9 is referred to as a distal end area Ar1. Furthermore, an area that continues toward the proximal end side A2 from the distal end area Ar1 is referred to as another area Ar2. In addition, the distal end portion 62 is provided, on the surface of the base 61 that faces the treatment portion 811, in the entire of the distal end area Ar1. In contrast, the proximal end portion 63 is provided, on the surface of the base 61 that faces the treatment portion 811, in the entire of the other area Ar2.

The distal end portion 62 described above is constituted by a conductive property material made of, for example, aluminum or the like. In contrast, the proximal end portion 63 is constituted by a conductive property material made of, for example, stainless steel or the like. In other words, the distal end portion 62 is constituted by a material having higher electric conductivity than the proximal end portion 63.

As illustrated in FIG. 5, in the distal end portion 62 and the proximal end portion 63, a pad 64 made of a resin is mounted on a second treatment surface 60 that faces the treatment portion 811 in the state in which the pad 64 is laid across the distal end portion 62 and the proximal end portion 63. The pad 64 has the electrically insulation properties and thus has a function of preventing the jaw 6 and the vibration transmission member 8 from being short circuited. Furthermore, the pad 64 has a function of preventing, at the time of completion of incision of the target region LT performed by the ultrasound vibration, damage caused by the vibration transmission member 8 that is being operated ultrasound vibration coming into collision with the jaw 6.

Furthermore, the second treatment surface 60 corresponds to a treatment surface.

Configuration of Control Device

The control device 3 includes, as illustrated in FIG. 1, an ultrasound current supplying unit 31, a high frequency current supplying unit 32, and an energy control unit 33.

Here, as illustrated in FIG. 2, a pair of transducer purpose lead wires C1 and C1′ that constitute an electric cable C is joined to the ultrasound transducer 72.

Furthermore, the ultrasound current supplying unit 31 supplies alternating-current power to the ultrasound transducer 72 by way of the pair of the transducer purpose lead wires C1 and C1′ under the control of the energy control unit 33. As a result, the ultrasound transducer 72 produces ultrasound vibration.

Here, as illustrated in FIG. 2, in the transducer case 71, a first conductive portion 711 that extends from the end portion of the transducer case 71 on the proximal end side A2 toward the end portion of the transducer case 71 on the distal end side A1 is provided. Furthermore, although a specific illustration has been omitted, in the sheath 5, a second conductive portion that extends from the end portion of the sheath 5 on the proximal end side A2 toward the end portion of the sheath 5 on the distal end side A1 and that electrically connects the first conductive portion 711 and the base 61 is provided. In addition, a high frequency purpose lead wire C2 that constitutes an electric cable C is joined to the end portion of the first conductive portion 711 on the proximal end side A2. Furthermore, the high frequency purpose lead wire C2′ that constitutes the electric cable C is joined to the first mounting portion 731.

Then, the high frequency current supplying unit 32 supplies, under the control of the energy control unit 33, high frequency electrical power to a portion between the jaw 6 and vibration transmission member 8 by way of the pair of high frequency purpose lead wires C2 and C2′, the first conductive portion 711, the second conductive portion, and the horn 73. As a result, high frequency current flows in the target region LT that is gripped by the jaw 6 and the treatment portion 811. In other words, high frequency energy is applied to the target region LT. Then, Joule heat is generated in the target region LT as a result of the high frequency current flowing through the target region LT, and then, treatment is performed on the target region LT.

As described above, the jaw 6 and the vibration transmission member 8 have a function of a pair of electrodes.

Here, as described above, the distal end portion 62 is constituted by a material having higher electric conductivity than the proximal end portion 63. In other words, the structure is formed such that, if high frequency electrical power is supplied to a portion between the jaw 6 and the vibration transmission member 8, the high frequency current tends to more easily flow a portion between the distal end portion 62 and the treatment portion 811 than a portion between the proximal end portion 63 and the treatment portion 811. As a result, high frequency energy that is higher than high frequency energy applied to a region of the target region LT that is gripped by the end effector 9 at the other area Ar2 is applied to a region of the target region LT that is gripped by the end effector 9 at the distal end area Ar1. More specifically, in a first period of time after the high frequency energy is started to be applied to the target region LT, the high frequency energy is mainly applied to the region of the target region LT at the distal end area Ar1. Then, impedance in the region of the target region LT at the distal end area Ar1 is increased, and, if the high frequency current is difficult to flow in the region of the target region LT at the distal end area Ar1, in a second period of time that is subsequent to the first period of time, high frequency energy is applied to the region of the target region LT at the other area Ar2. In other words, in first period of time, a difference of the electric conductivity between the distal end portion 62 and the proximal end portion 63 is a difference of the electric conductivity that corresponds to an amount of, for example, about several tens of ohms that is an increase amount of the impedance in the distal end area Ar1.

The energy control unit 33 is, for example, a central processing unit (CPU), a field-programmable gate array (FPGA), or the like, and performs an operation of each of the ultrasound current supplying unit 31 and the high frequency current supplying unit 32 when the operation button 42 is pressed by the operator.

In the present embodiment, when the operation button 42 is pressed by the operator, the energy control unit 33 controls the operation of each of the ultrasound current supplying unit 31 and the high frequency current supplying unit 32, and causes the ultrasound energy and the high frequency energy to simultaneously be applied to the target region LT gripped by the end effector 9. Furthermore, in a specific period of time after the ultrasound energy and the high frequency energy are started to be applied, the energy control unit 33 causes the high frequency energy that is applied to the target region LT to be further increased than the high frequency energy that is applied in the other period of time. Furthermore, the specific period of time may be a period of time that is preset, or it may be possible to determine the end of the specific period of time by analyzing an image of the target region LT captured when the target region LT is being subjected to treatment.

According to the present embodiment described above, the following advantages are provided.

In the ultrasound treatment instrument 2 according to the present embodiment, the end effector 9 includes the distal end area Ar1 described above.

Therefore, it is possible to apply high frequency energy to the region of the target region LT that is gripped by the end effector 9 on the distal end side A1 and it is thus possible to improve the treatment property with respect to the region of the target region LT on the distal end side A1.

Furthermore, as a result of the end effector 9 including the distal end area Ar1, in a portion between the jaw 6 and the treatment portion 811, an amount of the high frequency current sneaking and flowing through the distal end side A1 of the distal end P1 of the end effector 9 is relatively increased. As a result, when the target region LT gripped by the end effector 9 is being subjected to treatment, it is possible to coagulate, by the high frequency current, a blood vessel or the like that is located on the distal end side A1 relative to the target region LT. In other words, an advantage is provided in that, when the target region LT is being subjected to treatment, it is possible to avoid incision of the blood vessel or the like caused by cavitation due to ultrasound vibration.

In particular, in the treatment system 1 according to the present embodiment, in a specific period of time after the treatment of the target region LT, the control device 3 causes the high frequency energy that is applied to the target region LT to be further increased than the high frequency energy that is applied in the other period of time. Accordingly, when the target region LT is being subjected to treatment, it is possible to coagulate the blood vessel or the like that is located on the distal end side A1 relative to the target region LT by the high frequency current in a short period of time, and, it is thus possible to preferably provide the advantages described above.

Furthermore, in the treatment system 1 according to the present embodiment, the control device 3 simultaneously applies the high frequency energy and the ultrasound energy to the target region LT from the end effector 9. As a result, it is possible to greatly reduce the treatment period of time as compared to the case in which only the high frequency energy is applied to the target region LT first and then the ultrasound energy is applied to the target region LT after elapse of a predetermined period of time.

In the following, another exemplary embodiment will be described.

In a description below, the same components as those of the embodiment described above are denoted by the same reference numerals, and detailed explanation thereof will be omitted or simplified.

In the present embodiment, the configuration of the end effector 9 described in the above embodiment is different. In a description below, for convenience of description, the end effector 9 according to the present embodiment is referred to as an end effector 9A.

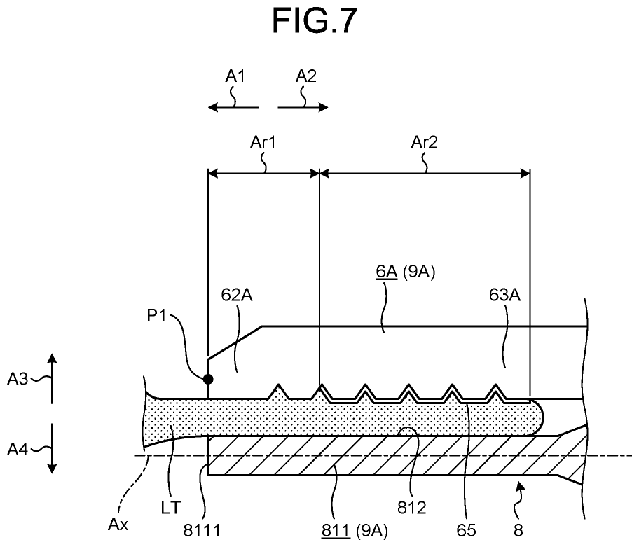

FIG. 7 is a diagram illustrating a configuration of the end effector 9 according to the present embodiment. Specifically, FIG. 7 is a cross-sectional view associated with that illustrated in FIG. 4.

In the end effector 9A, the configuration of the jaw 6 is different from that included in the end effector 9 described in the above embodiment. In a description below, for convenience of description, the jaw 6 according to the present embodiment is referred to as a jaw 6A.

In the jaw 6A, as illustrated in FIG. 7, the base 61, the distal end portion 62, and the proximal end portion 63 described in the above embodiment is constituted by a single member. In the following, for convenience of description, in the jaw 6A, a portion corresponding to the distal end portion 62 is referred to as a distal end portion 62A, and a portion corresponding to the proximal end portion 63 is referred to as a proximal end portion 63A.

In the end effector 9A according to the present embodiment, a coating layer 65 is formed on the surface of the proximal end portion 63A that faces the treatment portion 811. An example of the coating layer 65 includes a fluorine coat, a fluorine coat including conductive fillers, polyether ether ketone (PEEK) coat, or a polyimide (PI) coat.

In addition, the coating layer 65 functions as a resistance element of a high frequency current flowing in the target region LT. In other words, the structure is formed such that, if high frequency electrical power is supplied to a portion between the jaw 6A and the vibration transmission member 8, the high frequency current tends to more easily flow a portion between the distal end portion 62A and the treatment portion 811 than a portion between the proximal end portion 63A and the treatment portion 811. As a result, similarly to the embodiment described above, high frequency energy that is higher than high frequency energy applied to a region of the target region LT that is gripped by the end effector 9A at the other area Ar2 is applied to a region of the target region LT that is gripped by the end effector 9A at the distal end area Ar1.

Even when the end effector 9A according to the present embodiment is used, the same advantages as those provided in the embodiment described above are provided.

Furthermore, the same advantages as those described in the above embodiment are provided by simply providing the coating layer 65 to a general-purpose ultrasound treatment instrument, so that it is possible to reduce a cost.

In the following, another exemplary embodiment will be described.

In a description below, the same components as those of the first embodiment described above are denoted by the same reference numerals, and detailed explanation thereof will be omitted or simplified.

In the present embodiment, the configuration of the end effector 9 described above in the first embodiment is different. In a description below, for convenience of description, the end effector 9 according to the present embodiment is referred to as an end effector 9B.

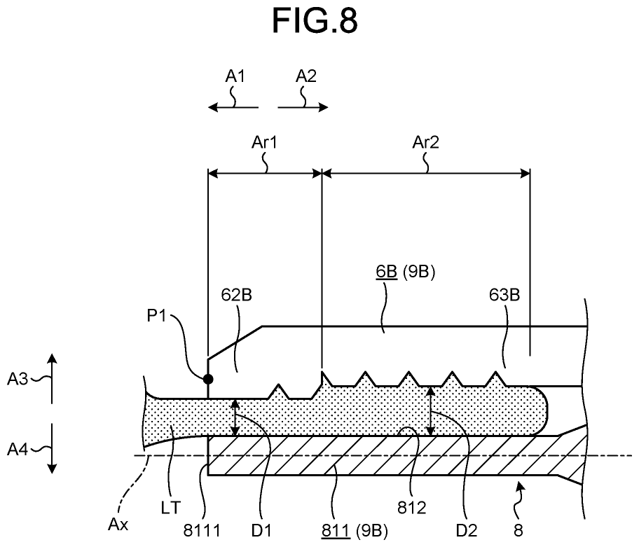

FIG. 8 is a diagram illustrating a configuration of the end effector 9B according to the present embodiment. Specifically, FIG. 8 is a cross-sectional view associated with that illustrated in FIG. 4.

In the end effector 9B, the configuration of the jaw 6 is different from that included in the end effector 9 described above in the first embodiment. In a description below, for convenience of description, the jaw 6 according to the present embodiment is referred to as a jaw 6B.

In the jaw 6B, as illustrated in FIG. 8, the base 61, the distal end portion 62, and the proximal end portion 63 described above in the first embodiment are constituted by a single member. In a description below, for convenience of description, in the jaw 6B, a portion corresponding to the distal end portion 62 is referred to a distal end portion 62B, and a portion corresponding to the proximal end portion 63 is referred to as a proximal end portion 63B.

In the end effector 9B according to the present embodiment, in the state in which the target region LT is gripped by the end effector 9B, a separation distance D1 between the distal end portion 62B and the treatment portion 811 is set to be larger than a separation distance D2 between the proximal end portion 63B and the treatment portion 811 (FIG. 8). As a result, a thickness of a region of the target region LT that is gripped by the end effector 9B at the distal end area Ar1 is smaller than a thickness of a region of the target region LT that is gripped by the end effector 9B at the other area Ar2, so that impedance exhibited in the region of the target region LT at the distal end area Ar1 is lower than impedance exhibited in the region of the target region LT at the other area Ar2. In other words, the structure is formed such that, if high frequency electrical power is supplied to a portion between the jaw 6B and the vibration transmission member 8, the high frequency tends to current more easily flow a portion between the distal end portion 62B and the treatment portion 811 than a portion between the proximal end portion 63B and the treatment portion 811. As a result, similarly to the first embodiment described above, high frequency energy that is higher than high frequency energy applied to the region of the target region LT that is gripped by the end effector 9B at the other area Ar2 is applied to the region of the target region LT that is gripped by the end effector 9B at the distal end area Ar1.

Even when the end effector 9B according to the present embodiment described above is used, the same advantages as those provided in the first embodiment are provided.

In the following, another exemplary embodiment will be described.

In a description below, the same components as those of the first embodiment described above are denoted by the same reference numerals, and detailed explanation thereof will be omitted or simplified.

In the present embodiment, the configuration of the end effector 9 described above in the first embodiment is different. In a description below, for convenience of description, the end effector 9 according to the present embodiment is referred to as an end effector 9C.

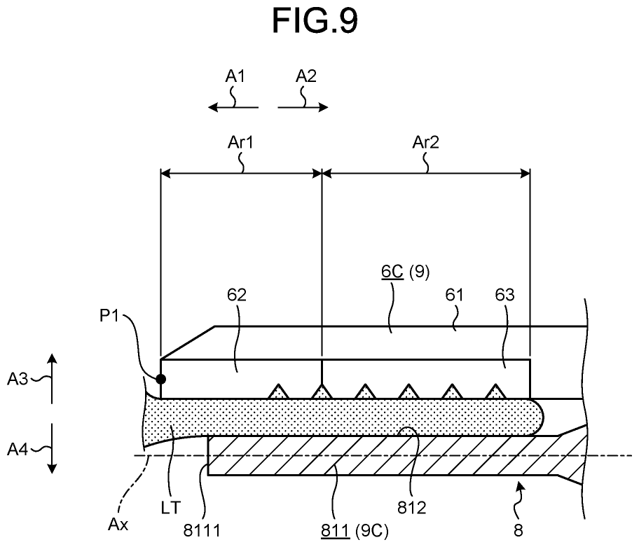

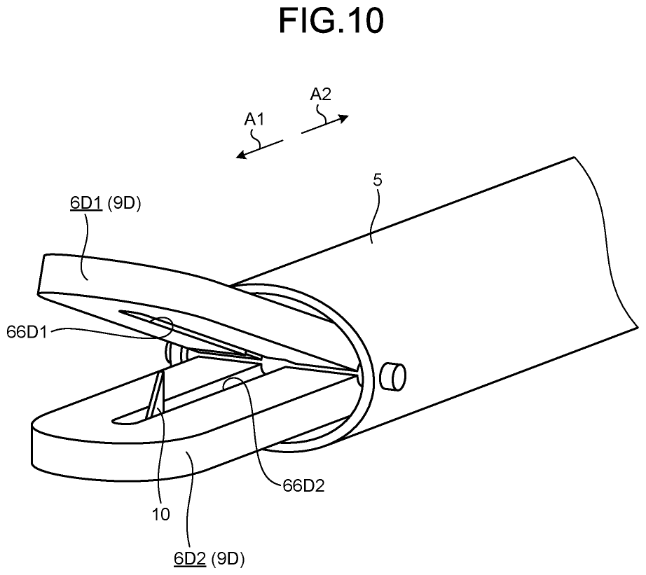

FIG. 9 is a diagram illustrating a configuration of the end effector 9C according to the present embodiment. Specifically, FIG. 9 is a cross-sectional view associated with that illustrated in FIG. 4.

In the end effector 9C, the length of the jaw 6 in the longitudinal direction is different from that included in the end effector 9 described above in the first embodiment. In a description below, for convenience of description, the jaw 6 according to the present embodiment is referred to as a jaw 6C.

As illustrated in FIG. 9, the length of the jaw 6C in the longitudinal direction is longer than that of the jaw 6 described above in the first embodiment. In other words, the jaw 6C further protrudes toward the distal end side A1 relative to the treatment portion 811 in the state in which the jaw 6C grips the target region LT with the treatment portion 811.

Even when the end effector 9C according to the present embodiment described above is used, the same advantages as those provided in the first embodiment are provided.

In the following, another exemplary embodiment will be described.

In a description below, the same components as those of the first embodiment described above are denoted by the same reference numerals, and detailed explanation thereof will be omitted or simplified.

In the present embodiment, the configuration of the end effector 9 described above in the first embodiment is different. In a description below, for convenience of description, the end effector 9 according to the present embodiment is referred to as an end effector 9D.

FIG. 10 is a diagram illustrating a configuration of the end effector 9D according to the present embodiment.

The end effector 9D performs treatment on the target region LT by gripping the target region LT and applying only the high frequency energy to the target region. As illustrated in FIG. 10, the end effector 9D includes a first and a second jaws 6D1 and 6D2.

In other words, in the present embodiment, a configuration for applying ultrasound energy to the target region LT (the vibration transmission member 8, the transducer unit 7, the ultrasound current supplying unit 31, etc.) are omitted.

The first jaw 6D1 has the same configuration as that described above in the first embodiment.

The second jaw 6D2 is constituted by a conductive property material and is fixed to the distal end of the sheath 5. Then, the target region LT is gripped by the first and the second jaws 6D1 and 6D2. Furthermore, if a high frequency current is supplied to a portion between the first and the second jaws 6D1 and 6D2, similarly to the first embodiment described above, high frequency energy that is higher than high frequency energy applied to a region of the target region LT that is gripped by the end effector 9D at the other area Ar2 is applied to a region of the target region LT that is gripped by the end effector 9D at the distal end area Ar1.

In addition, a cutter 10 is provided in the end effector 9D (FIG. 10).

Here, as illustrated in FIG. 10, in the first jaw 6D1, a first groove portion 66D1 that linearly extends from the proximal end of the first jaw 6D1 toward the distal end side A1 is provided on the surface that faces the second jaw 6D2. In contrast, similarly to the second jaw 6D2, a second groove portion 66D2 that linearly extends from the proximal end of the second jaw 6D2 toward the distal end side A1 is provided on the surface that faces the first jaw 6D1. Then, the cutter 10 is provided so as to be laid across the first and the second groove portions 66D1 and 66D2. Furthermore, the cutter 10 moves toward the distal end side A1 in accordance with an operation performed on an operation lever (not illustrated) by the operator. As a result, the cutter 10 is gripped by the end effector 9D and makes an incision in the target region LT coagulated by the applied high frequency energy.

Even when the end effector 9D according to the present embodiment described above is used, the same advantages as those provided in the first embodiment are provided.

In the above, detailed description of the preferred embodiments have been described; however, the disclosure is not limited to only the embodiments described above.

In the embodiments described above, the structure has been formed such that, by ingeniously designing the configuration of the jaws 6, 6A to 6C, and 6D1, high frequency energy that is higher than high frequency energy applied to the region of the target region LT at the other area Ar2 is applied to the region of the target region LT at the distal end area Ar1; however, the embodiments are not limited to this. For example, it may be possible to implement a structure in which high frequency energy that is higher than high frequency energy applied to the region of the target region LT at the other area Ar2 is applied to the region of the target region LT at the distal end area Ar1 by using the same configuration as that of the jaws 6, 6A to 6C, and 6D1 for the configuration of the treatment portion 811.

In the embodiment described above with respect to FIG. 7, the coating layer 65 is formed only on the surface of the proximal end portion 63A that faces the treatment portion 811; however, the embodiment is not limited to this. It may also be possible to provide a coating layer having higher electric conductivity than the coating layer 65 or provide a coating layer having a thinner film than the coating layer 65 on the surface of the distal end portion 62A that faces the treatment portion 811 as long as it is possible to implement a structure in which high frequency energy that is higher than high frequency energy applied to the region of the target region LT at the other area Ar2 is able to be applied to the region of the target region LT at the distal end area Ar1.

In the embodiments described above, it is possible to use a structure in which heat energy using a heater or the like may further be applied to the target region LT.

In the embodiments described above with respect to FIGS. 1-9, the shape of the treatment portion 811 is not limited to an octagon shape in cross section, but it may be possible to use another shape, such as a circular shape in cross section.

With the treatment instrument and the treatment system according to the disclosure, it is possible to effectively perform treatment on a living tissue on the distal end side of the end effector.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment instrument comprising:
a sheath extending along a central axis from a distal end to a proximal end; and
an end effector that is provided at the distal end of the sheath, the end effector being configured to grip a living tissue and apply high frequency energy to the living tissue in accordance with supplied electrical power to perform treatment on the living tissue, wherein
the end effector includes
a treatment surface for performing treatment on the living tissue, and
a pair of electrodes configured to grip the living tissue and apply the high frequency energy to the living tissue in accordance with the supplied electrical power,
the treatment surface includes
a distal end area that is provided on a distal end side of the treatment surface and that includes a distal end of the end effector, and
another area that is provided on a proximal end side of the treatment surface, and
the distal end area is configured to apply, to the living tissue, high frequency energy that is higher than high frequency energy applied by the other area, and
a first one of the pair of electrodes includes a distal end portion that is located in the distal end area and a proximal end portion that is located in the other area.

2. The treatment instrument according to claim 1, wherein the distal end portion is formed by a material having an electric conductivity that is higher than an electric conductivity of the proximal end portion.

3. The treatment instrument according to claim 1, wherein, the end effector includes a coating layer on a surface of the proximal end portion that is configured to come into contact with the living tissue.

4. The treatment instrument according to claim 1, wherein, in a state in which the living tissue is gripped by the end effector, a separation distance between the pair of electrodes in the other area is larger than a separation distance between the pair of electrodes in the distal end area.

5. The treatment instrument according to claim 1, wherein, in the end effector, a second one of the pair of electrodes is configured apply ultrasound energy to the living tissue to perform treatment on the living tissue.

6. The treatment instrument according to claim 5, wherein the first one of the pair of electrodes further protrudes toward a distal end relative to the second one of the pair of electrodes in a state in which the pair of electrodes grip the living tissue.

7. The treatment instrument according to claim 1, wherein the end effector is provided with a cutter configured to make an incision in the living tissue gripped by the end effector.

8. A treatment system comprising:
a treatment instrument configured to perform treatment on a living tissue; and
a control device configured to control an operation of the treatment instrument, wherein
the treatment instrument includes
a sheath extending along a central axis from a distal end to a proximal end, and
an end effector that is provided at the distal end of the sheath, the end effector being configured to grip the living tissue and apply high frequency energy to the living tissue in accordance with electrical power that is supplied from the control device to perform treatment on the living tissue,
the end effector includes
a treatment surface for performing treatment on the living tissue, and
a pair of electrodes configured to apply the high frequency energy to the living tissue,
the treatment surface includes
a distal end area that is provided on a distal end side of the treatment surface and that includes a distal end of the end effector, and
another area that is provided on a proximal end side of the treatment surface, and
the distal end area is configured to apply, to the living tissue, high frequency energy that is higher than high frequency energy applied by the other area, and
a first one of the pair of electrodes includes a distal end portion that is located in the distal end area and a proximal end portion that is located in the other area.

9. The treatment system according to claim 8, wherein
the pair of electrodes are configured to grip the living tissue and apply the high frequency energy to the living tissue in accordance with the electrical power supplied from the control device,
a second one of the pair of electrodes is configured to apply ultrasound energy to the living tissue to perform treatment on the living tissue, and
the control device is configured to cause the high frequency energy and the ultrasound energy to simultaneously be applied to the living tissue from the end effector.

10. The treatment system according to claim 8, wherein the control device is configured to cause the high frequency energy that is applied to the living tissue to be further increased in a specific period of time after the treatment on the living tissue is started relative to another period of time.

11. The treatment instrument according to claim 1, wherein
the distal end portion is formed of aluminum, and
the proximal end portion is formed of stainless steel.

12. The treatment instrument according to claim 3, wherein the coating layer is formed of one of a fluorine coat, a fluorine coat including electric conductive fillers, a polyether ether ketone (PEEK) coat, and a polyimide (PI) coat.

13. The treatment system according to claim 8, wherein the distal end portion is formed of a material having an electric conductivity that is higher than an electric conductivity of the proximal end portion.

14. A method of performing treatment on a living tissue by controlling electrical power supplied to an end effector configured to grip the living tissue, the end effector including a pair of electrodes configured to apply high frequency energy to the living tissue, a first one of the pair of electrodes including a distal end portion located in a distal end area and a proximal end portion located in another area, the method comprising:
applying, to the living tissue gripped by the distal end portion, energy that is higher than energy supplied to the living tissue gripped by the proximal end portion, to perform treatment on the living tissue.

15. The method according to claim 14, wherein the applying includes increasing an amount of high frequency energy that is applied to the living tissue gripped by the distal end portion in a specific period of time after the treatment performed on the living tissue is started, to perform the treatment on the living tissue.

16. The method according to claim 14, wherein the end effector is configured to supply, to a second one of the pair of electrodes included in the end effector, vibration due to ultrasound energy, and the applying includes
simultaneously applying the ultrasound energy and the high frequency energy to the living tissue gripped by the end effector, and
increasing the high frequency energy that is applied to the living tissue gripped by the distal end portion in a specific period of time after the ultrasound energy and the high frequency energy have started to be applied, to perform the treatment on the living tissue.
